(19) United States
(12) Patent Application Publication
(10) Pub. No.: US 2026/0176247 A1
(43) Pub. Date: Jun. 25, 2026

(54) SOLID STATE FORMS OF BELUMOSUDIL AND PROCESSES FOR PREPARATION THEREOF

(71) Applicant: ASSIA CHEMICAL INDUSTRIES LTD., Tel Aviv (IL)

(72) Inventors: Anchal SINGH, Himachal Pradesh (IN); Debashis DAS, Delhi (IN); Rinku SINGH, Noida Uttar Pradesh (IN); Annamalai SATHIYAMOORTHY, Tamil Nadu (IN); Sunil Dutt Keshav DURGAPAL, Gujarat (IN)

(21) Appl. No.: 19/129,103

(22) PCT Filed: Nov. 9, 2023
(86) PCT No.: PCT/IB2023/061339

§ 371 Date: May 12, 2025

(30) Foreign Application Priority Data
Nov. 11, 2022 (IN) 202211064677
Sep. 28, 2023 (IN) 202311065310

(51) Int. Cl. C07D 401/06; A61K 9/20

(52) U.S. Cl. CPC C07D 401/06; A61K 9/2054; C07B 2200/13

(57) ABSTRACT

The present disclosure encompasses a solid state form of Belumosudil, processes for preparation thereof, and pharmaceutical compositions thereof.

### FIELD OF THE DISCLOSURE

The present disclosure encompasses a solid state form of Belumosudil, processes for preparation thereof, and pharmaceutical compositions thereof.

### BACKGROUND OF THE DISCLOSURE

Belumosudil, 2-(3-(4-((1H-Indazol-5-yl)amino)quinazolin-2-yl)phenoxy)-N-isopropylacetamide, has the following chemical structure:

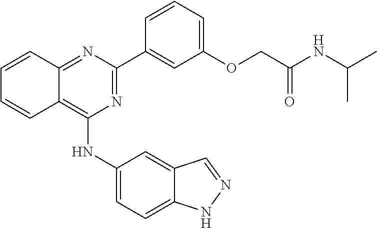 [SMILES: CC(C)NC(=O)COc1cccc(-c2nc(Nc3ccc4[nH]ncc4c3)c3ccccc3n2)c1]

Belumosudil is a ROCK2 inhibitor, and it is approved for the treatment of Chronic Graft-Versus-Host Disease.

The compound is described in International Publication No. WO 2006/105081. Crystalline forms of Belumosudil and salts thereof are described in International Publication Nos. WO 2021/129589, WO2022/020850, WO 2022/170864 and WO 2023/285706, as well as in CN114907325 and CN115073430. Processes for preparation of Belumosudil mesylate are described in IPCOM000270633, International Publication No. WO 2023/187697 and in “A process for the preparation of 2-{3-[4-(1H-indazol-5-ylamino)-2-quinazolinyl]phenoxy}-N-(propan-2-yl) acetamide or its salts”, Technical Disclosure Commons, (Aug. 31, 2023).

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Belumosudil.

### SUMMARY OF THE DISCLOSURE

The present disclosure provides a crystalline polymorph of Belumosudil, processes for preparation thereof, and pharmaceutical compositions thereof. This crystalline polymorph can be used to prepare other solid state forms of Belumosudil, Belumosudil salts and their solid state forms and preferably to prepare Belumosudil Mesylate and crystalline forms thereof.

The present disclosure also provides uses of the said solid state forms of Belumosudil in the preparation of other solid state forms of Belumosudil or salts thereof, and preferably to in the preparation of Belumosudil Mesylate and crystalline forms thereof.

The present disclosure provides crystalline polymorphs of Belumosudil for use in medicine, including for the treatment of Chronic Graft-Versus-Host Disease, Systemic Sclerosis, Idiopathic pulmonary fibrosis and/or Psoriasis vulgaris.

The present disclosure also encompasses the use of crystalline polymorphs of Belumosudil of the present disclosure for the preparation of pharmaceutical compositions and/or formulations.

In another aspect, the present disclosure provides pharmaceutical compositions comprising the crystalline polymorph of Belumosudil according to the present disclosure.

The present disclosure includes processes for preparing the above mentioned pharmaceutical compositions. The processes includes combining the crystalline polymorph of Belumosudil with at least one pharmaceutically acceptable excipient.

The crystalline polymorph of Belumosudil as defined herein and the pharmaceutical compositions or formulations of the crystalline polymorph of Belumosudil may be used as medicaments, such as for the treatment of Chronic Graft-Versus-Host Disease, Systemic Sclerosis, Idiopathic pulmonary fibrosis and/or Psoriasis vulgaris.

The present disclosure also provides methods of treating Chronic Graft-Versus-Host Disease, Systemic Sclerosis Idiopathic pulmonary fibrosis and/or Psoriasis vulgaris, by administering a therapeutically effective amount of the crystalline polymorph of Belumosudil of the present disclosure, or at least one of the above pharmaceutical compositions, to a subject suffering from Chronic Graft-Versus-Host Disease, Systemic Sclerosis, Idiopathic pulmonary fibrosis and/or Psoriasis vulgaris or otherwise in need of the treatment.

The present disclosure also provides uses of crystalline polymorph of Belumosudil of the present disclosure, or at least one of the above pharmaceutical compositions, for the manufacture of medicaments for treating e.g., Chronic Graft-Versus-Host Disease, Systemic Sclerosis, Idiopathic pulmonary fibrosis and/or Psoriasis vulgaris.

### BRIEF DESCRIPTION OF THE DRAWINGS

FIG. 1 shows an X-ray powder diffraction pattern (XRPD) of form B6 of Belumosudil obtained according to example 1, Procedure A.

FIG. 2 shows an X-ray powder diffraction pattern (XRPD) of form B6 of Belumosudil obtained according to example 1, Procedure B.

FIG. 3 shows a characteristic X-ray powder diffraction pattern (XRPD) of Belumosudil Mesylate Form M1.

### DETAILED DESCRIPTION OF THE DISCLOSURE

The present disclosure encompasses a solid state form of Belumosudil, processes for preparation thereof, and pharmaceutical compositions thereof.

Solid state properties of Belumosudil and crystalline polymorphs thereof can be influenced by controlling the conditions under which Belumosudil and crystalline polymorphs thereof are obtained in solid form.

The solid state form of Belumosudil as described in any aspect or embodiment of the present disclosure may be polymorphically pure, or substantially free of any other solid state (or polymorphic) forms.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Belumosudil described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Belumosudil. In some embodiments of the disclosure, the described crystalline polymorph of Belumosudil may contain from about 1% to about 20% (w/w), from about 5% to about 20% (w/w), or from about 5% to about 10% (w/w) of one or more other crystalline polymorph of the same Belumosudil.

Depending on which other crystalline polymorphs a comparison is made, the crystalline polymorphs of Belumosudil of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Belumosudil referred to herein as being characterized by graphical data “as depicted in” or “as substantially depicted in” a Figure will thus be understood to include any crystal forms of Belumosudil characterized with the graphical data having such small variations, as are well known to the skilled person, in comparison with the Figure.

As used herein, and unless stated otherwise, the term “anhydrous” in relation to crystalline forms of Belumosudil, relates to a crystalline form of Belumosudil which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Belumosudil of the present disclosure corresponds to a crystalline polymorph of Belumosudil that is physically separated from the reaction mixture in which it is formed.

As used herein, unless stated otherwise, the XRPD measurements are taken using copper Kα radiation wavelength 1.5418 Å. XRPD peaks reported herein are measured using CuKα radiation, λ=1.5418 Å, typically at a temperature of 25±3° C.

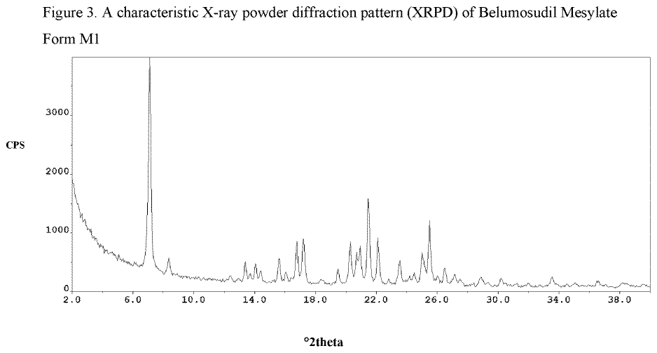

As used herein, crystalline form M1 of Belumosudil Mesylate refers to a crystalline form which may be characterized by X-ray powder diffraction pattern as depicted in FIG. 3. Crystalline form M1 of Belumosudil Mesylate may be characterized by XRPD peaks or the X-ray powder diffractogram disclosed in US 2023/0117059 or WO2022/020850.

In particular, crystalline form M1 of Belumosudil Mesylate may be characterized by data selected from one or more of the following: an X-ray powder diffraction pattern substantially as depicted in FIG. 3; an X-ray powder diffraction pattern having peaks at 7.1, 17.2, 20.3, 21.5 and 25.5 degrees 2-theta±0.2 degrees 2-theta; or an X-ray powder diffraction pattern having peaks at 7.1, 17.2, 20.3, 21.5 and 25.5 degrees 2-theta±0.2 degrees 2-theta, and also having any one, two, three, four or five additional peaks selected from 8.4, 15.5, 16.8, 19.5 and 22.1 degrees 2-theta±0.2 degrees 2-theta; or alternatively an X-ray powder diffraction pattern having peaks at 7.1, 8.4, 15.5, 16.8, 17.2, 19.5, 20.3, 21.5, 22.1 and 25.5 degrees 2-theta±0.2 degrees 2-theta.

Form M1 of Belumosudil Mesylate can be prepared by the processes described in WO2022/020850, or by crystallising Belumosudil Mesylate from a solvent comprising methanol or aqueous methanol. The crystallisation of Belumosudil Mesylate from a solvent comprising methanol, or aqueous methanol may comprise: preparing a mixture comprising Belumosudil mesylate in solvent selected from methanol, or methanol and water, and optionally seeds of Belumosudil mesylate, preferably seeds of Belumosudil mesylate form M1 (optionally at a temperature of about −15° C. to about 30° C.; or about −15° C. to about 20° C. or about −15° C. to about 10° C.; or about −15° C. to about 0° C. or about −5° C. to about 30° C.; or about 5° C. to about 30° C.; or about 15° C. to about 30° C.; or about −15° C. to about 20° C.; or about −5° C. to about 20° C.; or about 5° C. to about 20° C.); optionally cooling the mixture (preferably wherein the cooling is to about 15° C. to about −10° C.); optionally stirring the mixture preferably for a period of: about 30 minutes to about 10 hours, about 1 hour to about 8 hours, about 1 hour to about 6 hours, or about 1 to about 4 hours); optionally isolating the Belumosudil Mesylate; and optionally drying. The mixture comprising Belumosudil mesylate in solvent selected from methanol, or methanol and water, and optionally seeds of Belumosudil mesylate may be prepared by combining, in any order, Belumosudil and methanesulfonic acid, and optionally Belumosudil mesylate seeds, more preferably wherein the Belumosudil mesylate seeds are Form M1, in the solvent. The Belumosudil mesylate Form M1 may be isolated by any suitable procedure, preferably by filtration, centrifuge, or decantation, and more preferably by filtration.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The solid state form of Belumosudil as described in any aspect or embodiment of the present disclosure may be chemically pure, or substantially free of any other compounds.

A compound may be referred to herein as chemically pure or purified compound or as substantially free of any other compounds. As used herein, the terms “chemically pure” or “purified” or “substantially free of any other compounds” refer to a compound that is substantially free of any impurities including enantiomers of the subject compound, diastereomers or other isomers. A chemically pure or purified compound or a compound that is substantially free of any other compound will be understood to mean that it contains about 10% (w/w) or less, about 5% (w/w) or less, about 4% (w/w) or less, about 3% (w/w) or less, about 2% (w/w) or less, about 1.5% (w/w) or less, about 1% (w/w) or less, about 0.8% (w/w) or less, about 0.6% (w/w) or less, about 0.4% (w/w) or less, about 0.2% (w/w) or less, about 0.1% (w/w) or less, or about 0% of any other compound as measured, for example, by HPLC. Alternatively, a chemically pure or purified compound or a compound that is substantially free of any other compound will be understood to mean that it contains about 10% area percent or less, about 5% area percent or less, about 4% area percent or less, about 3% area percent or less, about 2% area percent or less, about 1.5% area percent or less, about 1% area percent or less, about 0.8% area percent or less, about 0.6% area percent or less, about 0.4% area percent or less, about 0.2% area percent or less, about 0.1% area percent or less, or about 0% of any other compound as measured by HPLC.

Thus, pure or purified Belumosudil described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2% (w/w), greater than about 99.4% (w/w), greater than about 99.6% (w/w), greater than about 99.8% (w/w), greater than about 99.9% (w/w), or about 100% of the subject Belumosudil. Alternatively, pure or purified Belumosudil, described herein as substantially free of any compounds would be understood to contain greater than about 90% area percent, greater than about 95% area percent, greater than about 96% area percent, greater than about 97% area percent, greater than about 98% area percent, greater than about 98.5% area percent, greater than about 99% area percent, greater than about 99.2% area percent, greater than about 99.4% area percent, greater than about 99.6% area percent, greater than about 99.8% area percent, greater than about 99.9% area percent, or about 100% of the subject Belumosudil.

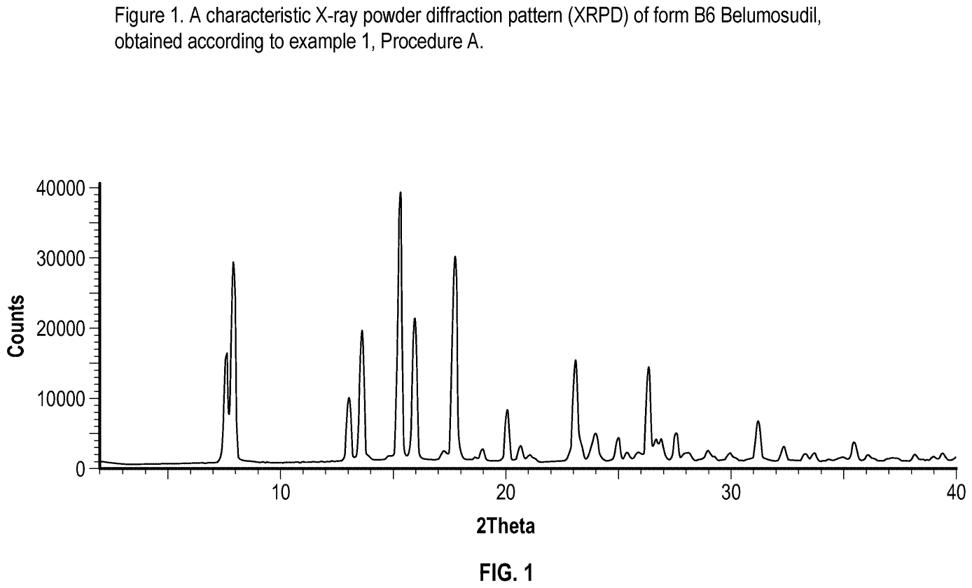

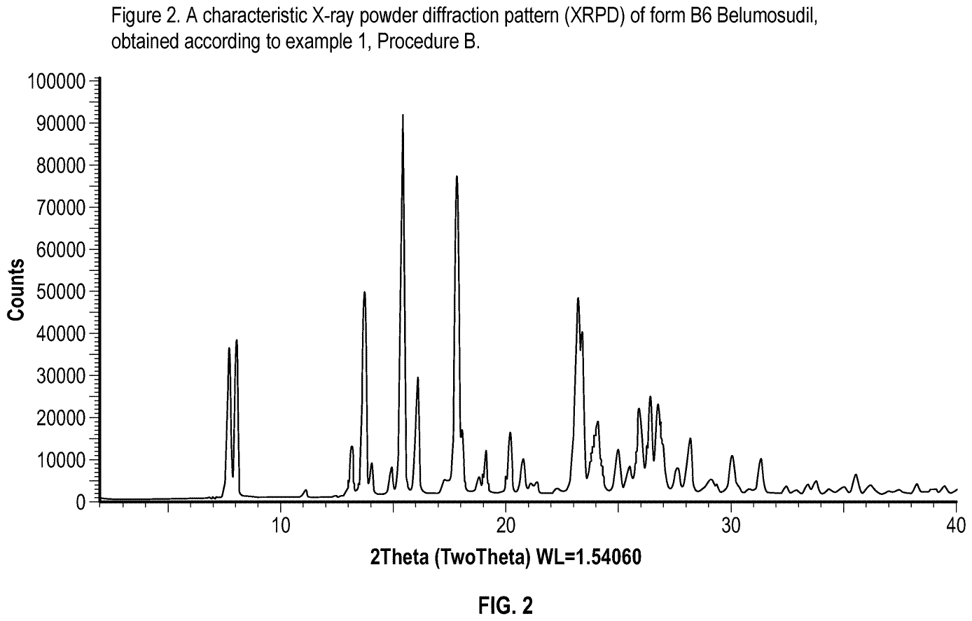

The present disclosure includes a crystalline polymorph of Belumosudil, designated form B6. The crystalline Form B6 of Belumosudil may be characterized by data selected from one or more of the following: an X-ray powder diffraction pattern substantially as depicted in FIG. 1 or FIG. 2; an X-ray powder diffraction pattern having peaks at 7.9, 13.0, 13.6, 20.1 and 26.3 degrees 2-theta±0.2 degrees 2-theta; and combinations of these data.

Crystalline Form B6 of Belumosudil may be further characterized by an X-ray powder diffraction pattern having peaks at 7.9, 13.0, 13.6, 20.1 and 26.3 degrees 2-theta±0.2 degrees 2-theta, and also having any one, two, three or four additional peaks selected from 15.3, 16.0, 23.1 and 31.2 degrees 2-theta±0.2 degrees 2-theta.

Crystalline Form B6 of Belumosudil may be alternatively characterized by an X-ray powder diffraction pattern having peaks at 7.9, 13.0, 13.6, 15.3, 16.0, 20.1, 23.1, 26.3 and 31.2 degrees 2-theta±0.2 degrees 2-theta.

According to any aspect or embodiment of the process crystalline Form B6 of Belumosudil may be further characterized by an X-ray powder diffraction pattern having an absence of peaks at any one of the following (i), (ii), and (iii), or a combination of any two, three or four of the following: (i), (ii), and (iii):
(i) 3.5 to 6.5 degrees 2-theta±0.2 degrees 2-theta; or 2.5 to 6.5 degrees 2-theta±0.2 degrees 2-theta; or 2.0 to 7.1 degrees 2-theta±0.2 degrees 2-theta;
(ii) 9.0 to 10.0 degrees 2-theta±0.2 degrees 2-theta; or 8.6 to 10.4 degrees 2-theta 0.2 degrees 2-theta; or 8.4 to 10.6 degrees 2-theta±0.2 degrees 2-theta;
(iii) 11.8 to 12.2 degrees 2-theta±0.2 degrees 2-theta; or 11.6 to 12.4 degrees 2-theta±0.2 degrees 2-theta.

In one embodiment of the present disclosure, crystalline Form B6 of Belumosudil is isolated. Particularly, crystalline Form B6 of Belumosudil according to any aspect or embodiment of the disclosure may be isolated

In any aspect or embodiment crystalline Form B6 of Belumosudil may be polymorphically pure.

In any aspect or embodiment, crystalline Form B6 of Belumosudil may be chemically pure.

In any aspect or embodiment, crystalline Form B6 of Belumosudil may be a methanol solvate. Alternatively, according to any aspect or embodiment of the present disclosure, crystalline form B6 of Belumosudil may contain about 6% to about 8% of methanol, preferably about 7% of methanol by weight.

Crystalline Form B6 of Belumosudil may be characterized by each of the above characteristics alone/or by all possible combinations, e.g., an XRPD pattern having peaks at 7.9, 13.0, 13.6, 20.1 and 26.3 degrees 2-theta±0.2 degrees 2-theta; an XRPD pattern as depicted in FIG. 1 or in FIG. 2, and combinations thereof.

The above crystalline polymorph can be used to prepare other crystalline polymorphs of Belumosudil, Belumosudil salts and their solid state forms, preferably Belumosudil mesylate and crystalline forms thereof.

The present disclosure encompasses a process for preparing other solid state forms of Belumosudil, Belumosudil salts and their solid state forms thereof. The process includes preparing the solid state form of Belumosudil by the processes of the present disclosure, and converting that form to a different form of Belumosudil or to a salt Belumosudil, preferably acid addition salts and more preferably Belumosudil mesylate.

The present disclosure relates to processes for preparation of Belumosudil and crystalline form thereof, preferably form B6. The processes of the present disclosure may further comprises a step of conversion of Belumosudil, preferably form B6 of Belumosudil, to Belumosudil Mesylate. The process may comprise reacting Belumosudil Form B6 as described in any aspect or embodiment, with methanesulfonic acid, and optionally isolating Belumosudil Mesylate.

The present disclosure further provides a process for the preparation of Belumosudil, particularly Form B6 of Belumosudil, wherein the process comprises reacting 1,1-dimethylethyl 5-[[2-[3-[2-[(1-methylethyl)amino]-2-oxoethoxy]-phenyl]4-quinazolinyl]amino]-1H-indazole-1-carboxylate with cesium carbonate. The process may particularly comprise: (a) reacting 1,1-dimethylethyl 5-[[2-[3-[2-[(1-methylethyl)amino]-2-oxoethoxy]phenyl]-4-quinazolinyl]amino]-1H-indazole-1-carboxylate with cesium carbonate in one or more solvents, preferably one or more organic solvents; preferably with heating; and (b) optionally isolating Belumosudil from the reaction mixture. In any aspect or embodiment, step (a) may be carried out in an inert atmosphere, preferably under a nitrogen atmosphere. Step (a) may be carried out in one or more polar solvents. The one or more polar solvent is preferably one or more alcohols (optionally wherein the alcohol is a C₁ to C₆ alcohol, or a C₁₋C₃ aliphatic alcohol, and particularly methanol); or a mixture of one or more alcohols (optionally wherein the alcohol is a C₁ to C₆ alcohol, or a C₁₋C₃ aliphatic alcohol, and particularly methanol) and a polar aprotic solvent (optionally DMSO, THF, DMF, and particularly DMSO). Preferably, according to any aspect or embodiment of this process, step (a) is carried out in methanol, or a mixture of methanol and DMSO). In any aspect or embodiment, step (a) may be carried out at a temperature of: about 20° C. to about 120° C.; about 20° C. to about 115° C.; about 20° C. to about 110° C.; about 20° C. to about 100° C.; or about 20° C. to about 90° C.; or at about the reflux temperature of the mixture. Particularly, according to any aspect or embodiment of the process, the solvent in step (a) may be methanol, and the reaction may be carried out at a temperature of: about 50° C. to about 120° C.; about 55° C. to about 115° C.; about 60° C. to about 110° C.; 65° C. to about 90° C.; or about 70° C. to about 100° C.; or at about the reflux temperature of the mixture. More particularly, step (a) is preferably carried out in methanol at reflux temperature. Alternatively, according to any aspect or embodiment of the process, the solvent in step (a) may be a mixture of methanol and DMSO, and the reaction may be carried out at a temperature of: 20° C. to about 50° C.; about 25° C. to about 45° C.; about 25° C. to about 40° C.; or preferably about 30° C. to about 35° C. In any aspect or embodiment, the amount of cesium carbonate in step (a) may be about 0.12 mole equivalents to about 1 equivalent, preferably about 0.4 equivalents. to about 0.6 equivalents relative to 1,1-dimethylethyl 5-[[2-[3-[2-[(1-methylethyl)-amino]-2-oxoethoxy]phenyl]-4-quinazolinyl]amino]-1H-indazole-1-carboxylate. In any aspect or embodiment, the total solvent in step (a) is in an amount of: about 5 to about 50 ml, about 5 to about 40 ml or about 5 to about 30 ml, or about 5 to about 25 ml, preferably about 7 to about 20 ml per gram of 1,1-dimethylethyl 5-[[2-[3-[2-[(1-methylethyl)amino]-2-oxoethoxy]phenyl]-4-quinazolinyl]amino]-1H-indazole-1-carboxylate. In any aspect or embodiment, the reaction mixture may be purged with nitrogen. According to any aspect or embodiment of the process, the reaction mixture may be filtered. In any aspect or embodiment, step (b) can comprise precipitating the Belumosudil. Optionally, according to any aspect or embodiment, step (b) may include a step of adding seed crystals to facilitate precipitation/crystallisation. According to any aspect or embodiment, step (b) may be carried out by addition of methanol, preferably wherein step (b) is carried out at a temperature of about 10° C. to about 35° C., about 15° C. to about 35° C.; or about 20° C. to about 30° C.; or preferably about 30° C. and optionally adding seeds of Belumosudil base. Alternatively, Belumosudil may be precipitated in step (b) by: evaporating at least part of the solvent from the reaction mixture, and optionally cooling the reaction mixture. In any aspect or embodiment, the evaporating may be carried out at a temperature of: about 40° C. to about 80° C.; about 45° C. to about 70° C.; about 45° C. to about 60° C.; about 45° C. to about 55° C.; or about 50° C.; preferably wherein the evaporating is carried under reduced pressure, preferably at reduced pressure. The cooling may be to a temperature of: about −10° C. to about 40° C.; about 0° C. to about 35° C.; about 10° C. to about 30° C.; about 20° C. to about 28° C.; or about 25° C. The evaporating may be carried out to reduce the volume of the mixture by: about 30% to about 70%; about 40% to about 60%, about 45% to about 55%, of the original reaction mixture volume. In any aspect or embodiment of the process, step (b) may comprise: (i) evaporating at least part of the solvent from the reaction mixture, (ii) optionally cooling, (iii) adding a polar solvent (preferably a polar aprotic solvent, particularly dimethylsulfoxide, to form a solution, (iv) optionally filtering the solution, (v) evaporating at least part of the solvent or solvents from the solution (preferably to reduce the volume of the mixture by: about 30% to about 70%; about 40% to about 60%, about 45% to about 55%, or about 50%, of the original reaction mixture volume, (vi) optionally cooling; and (vii) adding one or more polar protic solvents to precipitate Belumosudil. The evaporating in step (v) may be carried out at a temperature of: about 40° C. to about 80° C.; about 45° C. to about 70° C.; about 45° C. to about 60° C.; about 45° C. to about 55° C.; or about 50° C.; preferably wherein the evaporating is carried under reduced pressure, preferably at reduced pressure. Preferably, the polar solvent in step (iii) may be used in an amount of: about 1 ml to about 10 ml, about 2 ml to about 8 ml or about 3 ml to about 6 ml, or about 4 ml per gram of 1,1-dimethylethyl 5-[[2-[3-[2-[(1-methylethyl)amino]-2-oxoethoxy]phenyl]-4-quinazolinyl]amino]-1H-indazole-1-carboxylate. Preferably, the polar protic solvent in step (vii) is an alcohol, optionally wherein the alcohol is a C₁ to C₆ alcohol, or a C₁₋C₃ aliphatic alcohol, or methanol. In any aspect or embodiment of the process, the cooling in step (ii) may be carried out, preferably to a temperature of: about −10° C. to about 40° C.; about 0° C. to about 35° C.; about 10° C. to about 30° C.; about 20° C. to about 28° C.; or about 25° C. Preferably, the cooling in step (vi) is carried out, more preferably to a temperature of: about −10° C. to about 40° C.; about 0° C. to about 35° C.; about 10° C. to about 30° C.; about 20° C. to about 28° C.; or about 25° C. According to any aspect or embodiment, the process may further comprise stirring the mixture in step (vii), preferably wherein the stirring is at a temperature of: about −10° C. to about 40° C.; about 0° C. to about 35° C.; about 10° C. to about 30° C.; about 20° C. to about 28° C.; or about 25° C. The stirring may be conducted for any suitable period of time to obtain the product, particularly: about 30 minutes to about 24 hours, about 45 minutes to about 18 hours, about 60 minutes to about 12 hours, about 80 minutes to about 6 hours, about 100 minutes to about 4 hours, about 120 minutes to about 240 minutes, or about 150 minutes. The process may further comprise isolating the Belumosudil, preferably by filtration. Particularly, the process may further comprise isolating and drying the Belumosudil, preferably wherein the drying is carried out at a temperature of: about 40° C. to about 80° C.; about 45° C. to about 70° C.; about 45° C. to about 65° C.; about 45° C. to about 60° C.; or about 50° C. to about 55° C.; preferably at reduced pressure. Preferably, the drying is carried out for a sufficient time to remove the solvents, more particularly the drying may be carried out over a period of: about 1 hour to about 36 hours, about 2 hours to about 28 hours, about 6 hours to about 18 hours, about 8 hours to about 12 hours, or about 10 hours.

In one embodiment the present disclosure relates to a process for preparation of Belumosudil, preferably form B6 of Belumosudil, wherein the process comprises reacting 1,1-Dimethylethyl 5-[[2-[3-[2-[(1-methylethyl)amino]-2-oxoethoxy]phenyl]-4-quinazolinyl]amino]-1H-indazole-1-carboxylate with cesium carbonate in the presence of one or more solvents, preferably at elevated temperature to afford Belumosudil and isolating Belumosudil, preferably form B6 of Belumosudil. As used herein, unless otherwise indicated the term “elevated” temperature refers to a temperature of about 30° C. to about 100° C., about 30° C. to about 80° C., or about 30° C. to about 50° C.

In a more particular embodiment the present disclosure relates to a process for preparation of Belumosudil, preferably form B6 of Belumosudil, wherein the process comprises: a) reacting 1,1-Dimethylethyl 5-[[2-[3-[2-[(1-methylethyl)amino]-2-oxoethoxy]phenyl]-4-quinazolinyl]amino]-1H-indazole-1-carboxylate with cesium carbonate in the presence of one or more solvents, preferably with heating; b) nitrogen purging; c) optionally concentrating the reaction mixture; d) optionally cooling; e) optionally adding a solvent, preferably DMSO, to obtain a solution; f) optionally filtering the reaction mixture; g) optionally concentrating the reaction mixture; h) optionally cooling the reaction mixture; i) optionally adding a solvent; j) filtering the obtained solid and washing to obtain Belumosudil, preferably form B6 of Belumosudil.

Step a) may be performed in a solvent system comprising one or more polar protic solvents, preferably one or more alcohols, more preferably methanol at reflux. Alternatively, step a) may be performed in a solvent system comprising one or more polar protic solvents, preferably one or more alcohols, more preferably methanol, and one or more polar-aprotic solvents, preferably DMSO. Any one of steps c) and g) may comprise concentrating the reaction mixture by any method known in the art and preferably by distillation. Any one of steps d) and h) may comprise cooling the reaction mixture to about room temperature. The process may comprise further washing and drying steps and optionally seeding with form B6.

In another embodiment the present disclosure relates to a process for preparation of Belumosudil, preferably form B6 of Belumosudil, wherein the process comprises A) reacting 1,1-Dimethylethyl 5-[[2-[3-[2-[(1-methylethyl)amino]-2-oxoethoxy]phenyl]-4-quinazolinyl]amino]-1H-indazole-1-carboxylate with cesium carbonate in methanol, DMSO or mixture thereof, B) optionally filtering the reaction mixture; C) optionally adding methanol and/or optionally seeding with form B6 seeds; D) cooling the reaction mixture to about room temperature; and E) optionally removing the solvent and optionally washing the obtained solid by slurrying the solid with methanol and/or water or mixture thereof to obtain Belumosudil, preferably form B6 of Belumosudil. Step E) may be repeated several times as necessary to remove residual DMSO. The process may include nitrogen purging and further solvent removal/concentration steps at any time during the process. In any embodiment, solvents may be removed by any method known in the art such as filtration, distillation etc. The obtained product may be dried.

In any embodiment of this process, the reaction in step (A) is preferably performed at 20° C. to about 50° C., about 30° C. to about 50° C. or at about 30° C. to about 40° C. In any aspect or embodiment, the reaction in step (A) is preferably performed in a mixture of methanol and DMSO. In any aspect or embodiment, the amount of cesium carbonate in step A) is about 0.12 mole equivalents to about 1 mole equivalent, preferably about 0.4 to about 0.6 mole equivalents relative to starting material 1,1-Dimethylethyl 5-[[2-[3-[2-[(1-methylethyl)amino]-2-oxoethoxy]phenyl]-4-quinazolinyl]amino]-1H-indazole-1-carboxylate. In any aspect or embodiment of this process the methanol in step A) may be present at an amount of about 5 to about 50 ml, about 10 to about 40 ml or about 15 to about 30 ml, preferably about 20 grams per gram of the starting material, 1,1-Dimethylethyl 5-[[2-[3-[2-[(1-methylethyl)amino]-2-oxoethoxy]phenyl]-4-quinazolinyl]amino]-1H-indazole-1-carboxylate. In any aspect or embodiment In any aspect or embodiment of this process the DMSO in step A) may be present at an amount of about 1 ml to about 10 ml, about 2 ml to about 8 ml or about 3 ml to about 6 ml, or about 4 ml per gram of the starting material, 1,1-Dimethylethyl 5-[[2-[3-[2-[(1-methylethyl)amino]-2-oxoethoxy]-phenyl]-4-quinazolinyl]amino]-1H-indazole-1-carboxylate. In any embodiment of this process, step (A) comprises stirring the mixture for a sufficient amount of time to Belumosudil.

In a particular embodiment the present disclosure relates to a process for preparation of Belumosudil, preferably form B6 of Belumosudil, wherein the process comprises a) reacting 1,1-Dimethylethyl 5-[[2-[3-[2-[(1-methylethyl)amino]-2-oxoethoxy]phenyl]-4-quinazolinyl]amino]-1H-indazole-1-carboxylate with cesium carbonate in methanol at about reflux temperature; b) nitrogen purging; c) concentrating the reaction mixture to about 3-4 vol; d) cooling the reaction mixture to about room temperature; e) adding DMSO to obtain a solution; f) cooling the reaction mixture to room temperature; g) concentrating the reaction mixture to about 5-6 vol; h) cooling the reaction mixture to about room temperature; i) adding methanol; j) filtering the obtained solid and washing to obtain Belumosudil, preferably form B6 of Belumosudil.

The present disclosure further provides a process for preparing Belumosudil Form B6 as defined in any aspect or embodiment, comprising crystallizing Belumosudil from a solvent comprising methanol. The process may comprise dissolving Belumosudil in methanol, preferably at elevated temperature, particularly at a temperature of: about 50° C. to about 120° C.; about 55° C. to about 115° C.; about 60° C. to about 110° C.; 65° C. to about 90° C.; or about 70° C. to about 100° C.; or at reflux temperature; and precipitating Belumosudil from the solution. The methanol may be used in an amount of: about 5 to about 50 ml, about 10 to about 40 ml or about 15 to about 30 ml, per gram of Belumosudil. In any aspect or embodiment of this process, the Belumosudil may be precipitated by evaporation preferably by partial evaporation, and/or cooling, optionally with seeding, preferably wherein the seeding is with Form B6 of Belumosudil. The evaporation or partial evaporation may be carried out for a sufficient time to reduce the volume of the mixture by: about 30% to about 70%; about 40% to about 60%, about 45% to about 55%, or about 50%, of the original volume. The evaporating can be carried out at a temperature of: about 40° C. to about 80° C.; about 45° C. to about 70° C.; about 45° C. to about 60° C.; about 45° C. to about 55° C.; or about 50° C.; preferably wherein the evaporating is carried under reduced pressure, preferably at reduced pressure. The cooling may be carried out to a temperature of: about −10° C. to about 40° C.; about 0° C. to about 35° C.; about 10° C. to about 30° C.; about 20° C. to about 28° C.; or about 25° C. According to any aspect or embodiment of this process, Belumosudil Form B6 may be prepared by a process comprising crystallizing Belumosudil from a solvent mixture selected from: methanol and tetrahydrofuran (THF); methanol and acetic acid; or methanol and dimethylsulfoxide (DMSO). Particularly the process may comprise: combining Belumosudil with a solvent selected from: THF, acetic acid or DMSO; preferably at temperature of about 20° C. to about 30° C., to form a solution; optionally filtering the solution; combining the solution with methanol to precipitate Belumosudil; optionally seeding; and optionally isolating Belumosudil Form B6. Preferably, when THF is used, it is preferably used in an amount of: about 5 to about 50 ml, about 10 to about 40 ml or about 15 to about 30 ml, or about 20 ml, per gram of Belumosudil. Preferably, when acetic acid is used, it is preferably used in an amount of: about 1 ml to about 15 ml, about 3 ml to about 10 ml; or about 5 m, per gram of Belumosudil. Particularly, when the solvent is DMSO, it is preferably used in an amount of: about 1 ml to about 15 ml, about 2 ml to about 10 ml; or about 3 ml to about 7 ml, per gram of Belumosudil. Preferably, prior to combining with methanol, the solution of Belumosudil in the solvent is filtered. The step of combining methanol with the solution of Belumosudil in the solvent, is preferably carried out by adding methanol to the solution. The methanol may be added in an amount of: about 10 ml to about 45 ml, about 15 ml to about 35 m, about 18 to about 32 ml, per gram of Belumosudil. Optionally the mixture may be seeded, preferably with Belumosudil Form B6 seeds. The mixture may be maintained, preferably with stirring, at a temperature of: about −10° C. to about 40° C.; about 0° C. to about 35° C.; about 10° C. to about 30° C.; about 20° C. to about 28° C.; or about 25° C. The mixture may be maintained, preferably with stirring, at this temperature for a suitable time to form Belumosudil B6, preferably about 30 minutes to about 24 hours; about 30 minutes to about 10 hours, about 45 minutes to about 5 hours, about 60 minutes to about 200 minutes. The process preferably comprises isolating the Belumosudil, preferably by filtration. The isolated produce may be dried, preferably, the drying may be carried out at a temperature of: about 40° C. to about 80° C.; about 45° C. to about 70° C.; about 45° C. to about 65° C., preferably at reduced pressure. The drying may be for any suitable period of time to remove the solvents. Preferably, the drying is carried out for a period of: about 1 hour to about 36 hours, about 4 hours to about 25 hours, about 6 hours to about 18 hours, about 8 hours to about 15 hours.

According to any aspect or embodiment the above processes may include a step of combining the product (i.e. Belumosudil Mesylate, preferably Belumosudil Mesylate Form M1; or Belumosudil, preferably Belumosudil Form B6) with at least one pharmaceutically acceptable excipient (for example as described below) to form a pharmaceutical composition.

In another aspect the disclosure provides a process for crystallizing Belumosudil form B6 from a mixture of methanol and DMSO.

In one embodiment, the disclosure provides a process for preparation of form B6 of Belumosudil comprising: i) dissolving Belumosudil in DMSO, preferably at about room temperature; ii) optionally filtering the reaction mixture; iii) adding methanol and optionally stirring; iv) optionally filtering the reaction mixture and optionally washing with methanol and/or water or mixture thereof. The process may comprise several washing steps and the obtained product may be dried, for example under vacuum to obtain form B6 of Belumosudil.

In any embodiment of this process, the solution in step (i) is preferably at room temperature. In any aspect or embodiment of this process the DMSO in step i) may be present at an amount of about 1 ml to about 10 ml, about 2 ml to about 9 ml or about 3 ml to about 8 ml per gram of Belumosudil, In any embodiment of this process, step (ii) comprises stirring the mixture, preferably about 1 to about 60 minutes, about 2 to about 30 minutes, about 2 to about 10 minutes, or about 5 minutes. In any aspect or embodiment of this process the methanol in step iii) may be present at an amount of about 10 to about 50 ml, about 15 to about 40 ml or about 20 to about 35 ml per gram of Belumosudil

In another aspect, the disclosure provides a process for crystallizing Belumosudil Mesylate form M1 from methanol or aqueous methanol.

In one embodiment the disclosure provides a process for the preparation of Belumosudil Mesylate, preferably form M1 of belumosudil mesylate wherein the process comprises: providing a mixture of Belumosudil base and methane sulfonic acid in methanol or aqueous methanol; seeding with form M1 seeds and crystallizing form M1. The reaction may be performed at a temperature range of about −15° C. to about 30° C. The solid material may be isolated by methods known in the art such as filtration and the process may comprise further washing and drying steps.

The processes described in the literature have significant disadvantages. International Publication No. WO 2006/105081 discloses a direct preparation of Belumosudil having a yield of 61% in the step for preparation of Belumosudil, and moreover requiring the use of column chromatography for purification of the product. This publication further discloses preparation of Belumosudil deprotection of 1,1-Dimethylethyl 5-[[2-[3-[2-[(1-methylethyl)-amino]-2-oxoethoxy]phenyl]-4-quinazolinyl]amino]-1H-indazole-1-carboxylate under acidic conditions (using trifluoroacetic acid, TFA) which affords the TFA salt of Belumosudil, and hence requires a further step in order to afford Belumosudil.

In contrast to the prior art processes, the processes of the present disclosure advantageously provide a convenient synthesis of Belumosudil and Belumosudil Mesylate from 1,1-Dimethylethyl 5-[[2-[3-[2-[(1-methylethyl)amino]-2-oxoethoxy]phenyl]-4-quinazolinyl]amino]-1H-indazole-1-carboxylate. Advantageously the processes of the present disclosure enable the production of highly pure Belumosudil (for example: having a purity of: at least 99.4%, at least 99.7%, at least 99.8%, or at least 99.9%). Surprisingly, the processes of the present disclosure are able to produce high purity Belumosudil without the need for column chromatography procedures or separate crystallization steps, which are undesirable on large scale particularly from the view of the use of large volumes of solvent and energy consumption (such as required for solvent evaporation from the chromatrography fractions), and/or result in a lower yield.

The present disclosure provides the above described crystalline polymorph of Belumosudil for use in the preparation of pharmaceutical compositions comprising Belumosudil, salts thereof and/or crystalline polymorphs thereof, preferably acid addition salts thereof and more preferably Belumosudil mesylate and/or crystalline forms thereof.

The present disclosure also encompasses the use of crystalline polymorph of Belumosudil, of the present disclosure for the preparation of pharmaceutical compositions of Belumosudil and/or solid state forms thereof

The present disclosure includes processes for preparing the above mentioned pharmaceutical compositions. The processes includes combining the crystalline polymorphs of Belumosudil, of the present disclosure with at least one pharmaceutically acceptable excipient.

Pharmaceutical combinations or formulations of the present disclosure contain the solid state forms of Belumosudil, of the present disclosure. In addition to the active ingredient, the pharmaceutical formulations of the present disclosure may contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Belumosudil and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Belumosudil can be administered. Belumosudil may be formulated for administration to a mammal, in embodiments to a human, by injection. Belumosudil can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Belumosudil and the pharmaceutical compositions and/or formulations of Belumosudil of the present disclosure can be used as medicaments, in embodiments in the treatment of Chronic Graft-Versus-Host Disease and/or Systemic Sclerosis.

The present disclosure also provides methods of treating Chronic Graft-Versus-Host Disease and/or Systemic Sclerosis by administering a therapeutically effective amount of any one or a combination of the crystalline polymorphs of Belumosudil of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

X-ray diffraction was performed on X-Ray powder diffractometer:
Bruker D8 Advance; Cu-Kα radiation (λ=1.5418 Å); Lynx eye detector; laboratory temperature 22-25° C.; PMMA specimen holder ring with silicon low background. Prior to analysis, the samples were gently ground by means of mortar and pestle in order to obtain a fine powder. The ground sample was adjusted into a cavity of the sample holder and the surface of the sample was smoothed by means of a cover glass.

### Measurement Parameters:

Scan range: 2-40 degrees 2-theta;
Scan mode: continuous;
Step size: 0.05 degrees;
Time per step: 0.5 s;
Sample spin: 30 rpm;
Sample holder: PMMA specimen holder ring with silicon low background.
All X-Ray Powder Diffraction peak values are calibrated with regard to standard silicon spiking in the sample.

### HPLC Method Used for Purity Analysis of Belumosudil and Belumosudil Mesylate

Column: C-18 (150*4.6 mm, 5μ)
Eluent-A: 0.1% ortho phosphoric acid in water
Eluent-B: Acetonitrile
Wavelength: 210 nm
Column Temperature: 40° C.
Flow rate: 1.0 mL/minute
Diluent: Trifluoroethanol+water+Acetonitrile
Method: Gradient

### Gradient:

| Time(min) | % A | % B |
|---|---|---|
| 0.0 | 85 | 15 |
| 3 | 85 | 15 |
| 20 | 70 | 30 |
| 30 | 30 | 70 |
| 40 | 30 | 70 |

### EXAMPLES

### Preparation of Starting Materials

Belumosudil and 5-[[2-[3-[2-[(1-methylethyl)amino]-2-oxoethoxy]phenyl]-4-quinazolinyl]amino]-1H-indazole-1-carboxylate can be prepared according to methods known from the literature, for example according to International Publication No. WO 2006/105081.

### Example 1: Preparation of Form B6 Belumosudil

### Procedure A

Belumosudil (3 grams) was taken in a 100 mL round bottom flask and was dissolved in 60 mL of THF at 25° C. The clear solution obtained was passed through micron filter and methanol (90 ml) was added. A clear solution was obtained. About 60 mg of Belumosudil seeds, obtained according to example 1, procedure B, were added at 25° C. The reaction mass was cooled to 10° C. and stirred for 2 hours. The solid was filtered off, washed with methanol (6 ml) at 10° C. and dried at 60° C. for 10 hours under vacuum. The obtained solid was analyzed by X-ray powder diffraction and the XRPD pattern is presented in FIG. 1.

### Procedure B

1,1-Dimethylethyl 5-[[2-[3-[2-[(1-methylethyl)amino]-2-oxoethoxy]phenyl]-4-quinazolinyl]amino]-1H-indazole-1-carboxylate (100 grams) and cesium carbonate (29.5 grams) were taken in a 5.0 L reactor, 2.0 L of Methanol was added and reaction mixture was refluxed for 3 hours. Then reaction progress was checked by HPLC. After the reaction complied, methanol was distilled off to half of its original volume at 50° C. under vacuum. The reaction mass was cooled to 25° C. and filtered off and washed at 25° C. with methanol (200 ml), water (500 ml) and methanol (200 ml) and dried at 55° C. under vacuum. The obtained solid was analyzed by XRD and designated as Belumosudil Form B6 and the XRPD pattern is presented in FIG. 2. Yield=84.3%, HPLC purity 99.7%

### Procedure C

Belumosudil (5 grams) was taken in a 100 mL round bottom flask and was dissolved in 25 mL of acetic acid at 20-30° C. A clear solution was obtained after stirring for 30 minutes. The solution was passed through micron filter and MeOH (100 ml) was added over 5 minutes and the reaction mixture was stirred further for about 2 hours at 25° C. The solid that precipitated out was filtered off, washed with 10 ml methanol at 25° C. and dried at 60° C. for 8 hours under vacuum. The obtained solid (4.5 grams) analyzed by XRPD and identified as Belumosudil Form B6

### Procedure D

Belumosudil (2.5 grams) was taken in a 100 mL round bottom flask and was dissolved in 16.25 mL of DMSO at 25-30° C. A clear solution was obtained within 5 minutes. The solution was passed through micron filter and MeOH (72.5 ml) was added at 20-30° C. Solid precipitated out during methanol addition and the reaction mixture was further stirred for 60 minutes and then it was filtered off, washed three times with methanol (5 ml+12.5 ml+5 ml) and dried for 8 hr at 60° C. under vacuum (1.9 grams). The obtained solid was analyzed by XRPD and identified as Belumosudil Form B6.

### Procedure E

Belumosudil (5 grams) was taken in a 100 mL round bottom flask and was dissolved in 15 mL of DMSO at 20-30° C. A clear solution was obtained. The solution was passed through micron filter and MeOH (100 ml) was added. Solid precipitated out during methanol addition and the reaction mixture was further stirred for 75 minutes and then it was filtered off, washed twice with methanol (10 ml each) and twice with water (25 ml each) and dried for 15 hours at 60° C. under vacuum. The obtained solid was analyzed by XRD and identified as Belumosudil Form B6.

### Procedure F

1,1-Dimethylethyl 5-[[2-[3-[2-[(1-methylethyl)amino]-2-oxoethoxy]phenyl]-4-quinazolinyl]amino]-1H-indazole-1-carboxylate (20 grams) and cesium carbonate (5.8 grams) were taken in a 1.0 L round bottom flask and was suspended in 400 mL of Methanol at reflux for 3 hours. Then reaction progress was monitored by HPLC. After reaction complied, purged nitrogen at 50-60° C. for 1 hour. Then the further reaction mixture was distilled off to 100 ml at 50° C. under vacuum. The reaction mixture was cooled to 25° C. and DMSO (80 ml) was added. Reaction mass was completely dissolved. It was passed through micron. Then the reaction mixture was distilled off to 150 ml at 50° C. under vacuum. The reaction mixture was cooled to 25-30° C. and Methanol (480 ml) was added. Solid precipitated out during methanol addition and the reaction mixture was further stirred for 150 minutes and then it was filtered off. Solid was filtered off, washed with methanol (40 ml), twice with water (100 ml each) and then again with methanol (40 ml) and dried at 50° C. for 10 hours under vacuum. The obtained solid was analyzed by XRPD and identified as Belumosudil Form B6. Yield=78.5%, HPLC purity 99.9%

### Example 2: Preparation of Form M1 of Belumosudil Mesylate

### Procedure A

Belumosudil form B6 (10.0 grams) was charged into reactor at 20-30° C. 0.5% aqueous methanol (150 mL) was charged into the reactor at 20-30° C. The reaction mass was stirred at 20-30° C. for 5-10 minutes. The reaction mass was cooled to −15° C. to 10° C. Belumosudil mesylate form M1 polymorph seed material (prepared according to International Publication No. WO2022/020850) and methane sulphonic acid (1 mole eq.) were charged to the reaction mass and it was stirred for ˜2.5 hours at −15° C. to 10° C. The solid was filtered and washed with 40 mL of 0.5% aqueous methanol. The obtained material was dried under vacuum to afford crystalline form M1 Belumosudil Mesylate. HPLC purity (>99.0%)

### Procedure B

Belumosudil form B6 (10.0 grams) was charged into reactor at 20-30° C. 0.5% aqueous methanol (150 mL) was charged into the reactor at 20-30° C. The reaction mass was stirred at 20-30° C. for 5-10 minutes. The reaction mass was cooled to 10° C. to 30° C. Belumosudil mesylate form M1 polymorph seed material (prepared according to International Publication No. WO2022/020850) and methane sulphonic acid (1 mole eq.) were charged to the reaction mass and it was stirred for 2.5 hours at 10-30° C. The solid was filtered under vacuum and the wet cake was washed with 40 mL of 0.5% aqueous methanol. The obtained material was dried under vacuum to afford crystalline form M1 of Belumosudil Mesylate. HPLC purity (>99.0%).

Further aspects and embodiments of the present disclosure are set out in the numbered clauses below
1. A crystalline form of Belumosudil, which may be designated form B6, which is characterized by an X-ray powder diffraction pattern having peaks at 7.9, 13.0, 13.6, 20.1 and 26.3 degrees 2-theta±0.2 degrees 2-theta.
2. A crystalline form of Belumosudil according to Clause 1, which is characterized by an X-ray powder diffraction pattern having peaks at 7.9, 13.0, 13.6, 20.1 and 26.3 degrees 2-theta±0.2 degrees 2-theta, and also having any one, two, three or four additional peaks selected from 15.3, 16.0, 23.1 and 31.2 degrees 2-theta±0.2 degrees 2-theta.
3. A crystalline form of Belumosudil according to Clause 1 or Clause 2, which is characterized by an X-ray powder diffraction pattern having peaks at 7.9, 13.0, 13.6, 15.3, 16.0, 20.1, 23.1, 26.3 and 31.2 degrees 2-theta±0.2 degrees 2-theta; or an X-ray powder diffraction pattern substantially as depicted in FIG. 1 or FIG. 2.
4. A crystalline form of Belumosudil according to any of Clauses 1, 2 and 3, which is further characterized by an X-ray powder diffraction pattern having an absence of peaks at 3.5 to 6.5 degrees 2-theta±0.2 degrees 2-theta.
5. A crystalline form of Belumosudil according to any of Clauses 1, 2 and 3, which is further characterized by an X-ray powder diffraction pattern having an absence of peaks at 2.5 to 6.5 degrees 2-theta±0.2 degrees 2-theta.
6. A crystalline form of Belumosudil according to any of Clauses 1, 2 and 3, which is further characterized by an X-ray powder diffraction pattern having an absence of peaks at 2.0 to 7.1 degrees 2-theta±0.2 degrees 2-theta.
7. A crystalline form of Belumosudil according to any of Clauses 1, 2, 3, 4, 5, and 6, which is further characterized by an X-ray powder diffraction pattern having an absence of peaks at 9.0 to 10.0 degrees 2-theta±0.2 degrees 2-theta.
8. A crystalline form of Belumosudil according to any of Clauses 1, 2, 3, 4, 5 and 6, which is further characterized by an X-ray powder diffraction pattern having an absence of peaks at 8.6 to 10.4 degrees 2-theta±0.2 degrees 2-theta.
9. A crystalline form of Belumosudil according to any of Clauses 1, 2, 3, 4, 5, and 6, which is further characterized by an X-ray powder diffraction pattern having an absence of peaks at 8.4 to 10.6 degrees 2-theta±0.2 degrees 2-theta.
10. A crystalline form of Belumosudil according to any of Clauses 1, 2, 3, 4, 5, 6, 7, 8 and 9, which is further characterized by an X-ray powder diffraction pattern having an absence of peaks at 11.8 to 12.2 degrees 2-theta±0.2 degrees 2-theta.
11. A crystalline form of Belumosudil according to any of Clauses 1, 2, 3, 4, 5, 6, 7, 8, and 9, which is further characterized by an X-ray powder diffraction pattern having an absence of peaks at 11.6 to 12.4 degrees 2-theta±0.2 degrees 2-theta.
12. A crystalline form of Belumosudil according to any of Clauses 1, 2, 3, 4, 5, 6, 7, 8, 9, 10, or 11, which is a methanol solvate, preferably containing about 6% to about 8% of methanol, preferably about 7% of methanol by weight.
13. A crystalline form of Belumosudil according to any of Clauses 1, 2, 3, 4, 5, 6, 7, 8, 9, 10, 11 or 12, which is isolated.
14. A crystalline form of Belumosudil according to any of Clauses 1, 2, 3, 4, 5, 6, 7, 8, 9, 10, 11, 12, or 13, which is substantially free of any other solid state forms; preferably containing about 20% (w/w) or less, about 10% (w/w) or less, about 5% (w/w) or less, about 2% (w/w) or less, about 1% (w/w) or less, or about 0% of any other forms of Belumosudil.
15. A pharmaceutical composition comprising a crystalline form of Belumosudil according to any of Clauses 1 to 14.
16. Use of a crystalline form of Belumosudil according to any of Clauses 1 to 14 for the preparation of a pharmaceutical composition and/or pharmaceutical formulation.
17. A pharmaceutical formulation comprising crystalline form of Belumosudil according to any of Clauses 1 to 14, or a pharmaceutical composition of Clause 15, with at least one pharmaceutically acceptable excipient.
18. A process for preparing a pharmaceutical formulation according to Clause 17, comprising combining a crystalline form of Belumosudil according to any of Clauses 1 to 14, or a pharmaceutical composition of Clause 15, with at least one pharmaceutically acceptable excipient.
19. A crystalline form of Belumosudil according to any of Clauses 1 to 14, or a pharmaceutical composition of Clause 15, or a pharmaceutical formulation according to Clause 17 for use as a medicament.
20. A crystalline form of Belumosudil according to any of Clauses 1 to 14, or a pharmaceutical composition of Clause 15, or a pharmaceutical formulation according to Clause 17 for use in the treatment of Chronic Graft-Versus-Host Disease, Systemic Sclerosis, Idiopathic pulmonary fibrosis and/or Psoriasis vulgaris.
21. A method of treating Chronic Graft-Versus-Host Disease, Systemic Sclerosis, Idiopathic pulmonary fibrosis and/or Psoriasis vulgaris, comprising administering a therapeutically effective amount of a crystalline form of Belumosudil according to any of Clauses 1 to 14, or a pharmaceutical composition of Clause 15, or a pharmaceutical formulation according to Clause 17 to a subject in need of the treatment.
22. A crystalline form of Belumosudil according to any of Clauses 1 to 14, or a pharmaceutical composition of Clause 15, or a pharmaceutical formulation according to Clause 17, for the manufacture of a medicament for the treatment of Chronic Graft-Versus-Host Disease, Systemic Sclerosis, Idiopathic pulmonary fibrosis and/or Psoriasis vulgaris.
23. Use of a crystalline form of Belumosudil according to any of Clauses 1 to 14, for the preparation of another solid state or crystalline polymorph form of: Belumosudil or Belumosudil salts or solid state forms thereof, preferably for preparing an acid addition salt of Belumosudil, and particularly for preparing Belumosudil mesylate and crystalline forms of Belumosudil mesylate.
24. A process for preparing other solid state forms of Belumosudil, Belumosudil salts and their solid state forms thereof comprising preparing crystalline Form B6 of Belumosudil according to any of Clauses 1 to 14, and converting the crystalline Form B6 to a different form of Belumosudil or to a salt Belumosudil, preferably an acid addition salt of Belumosudil, and more preferably Belumosudil mesylate.
25. A process according to Clause 24, for preparing Belumosudil Mesylate, comprising reacting Belumosudil Form B6 according to any of Clauses 1 to 14, with methanesulfonic acid, and optionally isolating Belumosudil Mesylate.
26. A process for the preparation of Belumosudil, preferably Form B6 of Belumosudil, wherein the process comprises (a) reacting 1,1-dimethylethyl 5-[[2-[3-[2-[(1-methylethyl)amino]-2-oxoethoxy]phenyl]-4-quinazolinyl]-amino]-1H-indazole-1-carboxylate with cesium carbonate, and optionally (b) isolating Belumosdil.
27. A process according to Clause 26, wherein the Belumosudil is Form B6 as defined in any of Clauses 1 to 14.
28. A process according to Clause 26 or Clause 27, which comprises:
   a) reacting 1,1-dimethylethyl 5-[[2-[3-[2-[(1-methylethyl)amino]-2-oxoethoxy]phenyl]-4-quinazolinyl]amino]-1H-indazole-1-carboxylate with cesium carbonate in one or more solvents, preferably one or more organic solvents; preferably with heating; and
   b) optionally isolating Belumosudil from the reaction mixture.
29. A process according to any of Clauses 26, 27 or 28, wherein step (a) is carried out under in an inert atmosphere, preferably wherein step (a) is carried out under nitrogen.
30. A process according to any of Clauses 26, 27, 28, or 29, wherein step a) is carried out in one or more polar solvents, or wherein step (a) is carried out in one or more polar protic solvents, preferably one or more alcohols, optionally wherein the alcohol is a C₁ to C₆ alcohol, or a C₁-C₃ aliphatic alcohol, or methanol; or wherein step (a) is carried out in a mixture of one or more alcohols (optionally wherein the alcohol is a C₁ to C₆ alcohol, or a C₁-C₃ aliphatic alcohol, and particularly methanol) and a polar aprotic solvent (optionally DMSO, THF, DMF, and particularly DMSO).
31. A process according to any of Clauses 26, 27, 28, 29, or 30, wherein step (a) is carried out in methanol or a mixture of methanol and DMSO.
32. A process according to any of Clauses 26, 27, 28, 29, 30, or 31, wherein step (a) is carried out at a temperature of: about 20° C. to about 120° C.; about 20° C. to about 115° C.; about 20° C. to about 110° C.; about 20° C. to about 100° C.; or about 20° C. to about 90° C.; or at about the reflux temperature of the mixture.
33. A process according to any of Clauses 26, 27, 28, 29, 30, 31, or 32, wherein the solvent in step (a) is methanol, and the reaction is carried out at a temperature of: about 50° C. to about 120° C.; about 55° C. to about 115° C.; about 60° C. to about 110° C.; 65° C. to about 90° C.; or about 70° C. to about 100° C.; or at about the reflux temperature of the mixture.
34. A process according to any of Clauses 26, 27, 28, 29, 30, 31, 32, or 33, wherein the solvent in step (a) is a mixture of methanol and DMSO, and the reaction is carried out at a temperature of: 20° C. to about 50° C.; about 25° C. to about 45° C.; about 25° C. to about 40° C.; or preferably about 30° C. to about 35° C.
35. A process according to any of Clauses 26, 27, 28, 29, 30, 31, 32, 33, or 34, wherein the amount of cesium carbonate in step (a) is about 0.12 mole equivalents to about 1 equivalent, preferably about 0.4 mole equivalents to about 0.6 mole equivalents, relative to 1,1-dimethylethyl 5-[[2-[3-[2-[(1-methylethyl)amino]-2-oxoethoxy]phenyl]-4-quinazolinyl]amino]-1H-indazole-1-carboxylate.
36. A process according to any of Clauses 26, 27, 28, 29, 30, 31, 32, 33, 34, or 35, wherein the total solvent in step (a) is in an amount of: about 5 to about 50 ml, about 5 to about 40 ml or about 5 to about 30 ml, or about 5 to about 20 ml, preferably about 7 to about 20 ml, per gram of 1,1-dimethylethyl 5-[[2-[3-[2-[(1-methylethyl)amino]-2-oxoethoxy]phenyl]-4-quinazolinyl]amino]-1H-indazole-1-carboxylate.
37. A process according to any of Clauses 26, 27, 28, 29, 30, 31, 32, 33, 34, 35, or 36, wherein following the reaction, the reaction mixture is filtered.
38. A process according to any of Clauses 26, 27, 28, 29, 30, 31, 32, 33, 34, 35, 36, or 37, wherein after the reaction, the reaction mixture is purged with nitrogen.
39. A process according to any of Clauses 26, 27, 28, 29, 30, 31, 32, 33, 34, 35, 36, 37, or 38, wherein step (b) comprises precipitating the Belumosudil.
40. A process according to any of Clauses 26, 27, 28, 29, 30, 31, 32, 33, 34, 35, 36, 37, 38, or 39, wherein step (b) comprises addition of seed crystals of Belumosudil.
41. A process according to any of Clauses 26, 27, 28, 29, 30, 31, 32, 33, 34, 35, 36, 37, 38, 39, or 40, wherein step (b), wherein step (b) comprises combining the reaction mixture with methanol, preferably wherein step (b) is carried out at a temperature of about 10° C. to about 35° C., about 15° C. to about 35° C.; or about 20° C. to about 30° C.; or preferably about 30° C.
42. A process according to any of Clauses 26, 27, 28, 29, 30, 31, 32, 33, 34, 35, 36, 37, 38, 39, 40, or 41, wherein Belumosudil isolated by a precipitation process comprising:
   evaporating at least part of the solvent from the reaction mixture, and
   optionally cooling the reaction mixture.
43. A process according to Clause 42, wherein the evaporating is carried out at a temperature of: about 40° C. to about 80° C.; about 45° C. to about 70° C.; about 45° C. to about 60° C.; about 45° C. to about 55° C.; or about 50° C.; preferably wherein the evaporating is carried under reduced pressure, preferably at reduced pressure.
44. A process according to Clause 42 or Clause 43, wherein the cooling is to a temperature of: about −10° C. to about 40° C.; about 0° C. to about 35° C.; about 10° C. to about 30° C.; about 20° C. to about 28° C.; or about 25° C.
45. A process according to any of Clauses 42, 43 or 44, wherein the evaporating is carried out to reduce the volume of the mixture by: about 30% to about 70%; about 40% to about 60%, about 45% to about 55%, of the original reaction mixture volume.
46. A process according to any of Clauses 42, 43, 44, or 45, wherein step (b) comprises:
   (i) evaporating at least part of the solvent from the reaction mixture,
   (ii) optionally cooling,
   (iii) adding a polar solvent, preferably a polar aprotic solvent, to form a solution,
   (iv) optionally filtering the solution,
   (v) evaporating at least part of the solvent or solvents from the solution,
   (vi) optionally cooling; and
   (vii) adding one or more polar protic solvents to precipitate Belumosudil.
47. A process according to Clause 46, wherein the evaporating is carried out to reduce the volume of the mixture by: about 30% to about 70%; about 40% to about 60%, about 45% to about 55%, or about 50%, of the original reaction mixture volume.
48. A process according to Clause 46 or Clause 47, wherein the evaporating is carried out at a temperature of: about 40° C. to about 80° C.; about 45° C. to about 70° C.; about 45° C. to about 60° C.; about 45° C. to about 55° C.; or about 50° C.; preferably wherein the evaporating is carried under reduced pressure, preferably at reduced pressure.
49. A process according to any of Clauses 46, 47 or 48, wherein the polar solvent in step (iii) is a polar aprotic solvent, preferably dimethylsulfoxide (DMSO).
50. A process according to any of Clauses 46, 47, 48, or 49, wherein the polar solvent in step (iii) is used in an amount of: about 1 ml to about 10 ml, about 2 ml to about 8 ml or about 3 ml to about 6 ml, or about 4 ml per gram of 1,1-dimethylethyl 5-[[2-[3-[2-[(1-methylethyl)amino]-2-oxoethoxy]phenyl]-4-quinazolinyl]amino]-1H-indazole-1-carboxylate.
51. A process according to any of Clauses 46, 47, 48, 48, or 50, wherein the polar protic solvent in step (vii) is an alcohol, optionally wherein the alcohol is a C₁ to C₆ alcohol, or a C₁-C₃ aliphatic alcohol, or methanol.
52. A process according to any of Clauses 46, 47, 48, 49, 50, or 51, wherein the cooling in step (ii) is carried out, preferably to a temperature of: about −10° C. to about 40° C.; about 0° C. to about 35° C.; about 10° C. to about 30° C.; about 20° C. to about 28° C.; or about 25° C.
53. A process according to any of Clauses 46, 47, 48, 49, 50, 51, or 52, wherein the cooling in step (vi) is carried out, preferably to a temperature of: about −10° C. to about 40° C.; about 0° C. to about 35° C.; about 10° C. to about 30° C.; about 20° C. to about 28° C.; or about 25° C.
54. A process according to any of Clauses 46, 47, 48, 49, 50, 51, 52, or 53, further comprising stirring the mixture in step (vii), preferably wherein the stirring is at a temperature of: about −10° C. to about 40° C.; about 0° C. to about 35° C.; about 10° C. to about 30° C.; about 20° C. to about 28° C.; or about 25° C.
55. A process according to Clause 54, wherein the stirring is conducted for about 30 minutes to about 24 hours, about 45 minutes to about 18 hours, about 60 minutes to about 12 hours, about 80 minutes to about 6 hours, about 100 minutes to about 4 hours, about 120 minutes to about 240 minutes, or about 150 minutes.
56. A process according to any of Clauses 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55, comprising isolating the Belumosudil, preferably by filtration.
57. A process according to any of Clauses 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56, comprising isolating and drying the Belumosudil, preferably wherein the drying is carried out at a temperature of: about 40° C. to about 80° C.; about 45° C. to about 70° C.; about 45° C. to about 65° C.; about 45° C. to about 60° C.; or about 50° C. to about 55° C.; preferably at reduced pressure.
58. A process according to Clause 57, wherein the drying is carried out over a period of: about 1 hour to about 36 hours, about 2 hours to about 28 hours, about 6 hours to about 18 hours, about 8 hours to about 12 hours, or about 10 hours.
59. A process for preparing Belumosudil Form B6 as defined in any of Clauses 1 to 14, comprising crystallizing Belumosudil from a solvent comprising methanol.
60. A process according to Clause 59, comprising crystallizing Belumosudil from methanol.
61. A process according to Clause 60, comprising dissolving Belumosudil in methanol, preferably at elevated temperature, particularly at a temperature of: about 50° C. to about 120° C.; about 55° C. to about 115° C.; about 60° C. to about 110° C.; 65° C. to about 90° C.; or about 70° C. to about 100° C.; or at reflux temperature; and precipitating Belumosudil from the solution.
62. A process according to any of Clauses 59, 60 or 61, wherein the methanol is used in an amount of: about 5 to about 50 ml, about 10 to about 40 ml or about 15 to about 30 ml, per gram of Belumosudil.
63. A process according to Clause 61 or Clause 62, wherein the Belumosudil is precipitated by evaporation preferably by partial evaporation, and/or cooling, optionally with seeding, preferably wherein the seeding is with Form B6 of Belumosudil.
64. A process according to Clause 63, wherein the evaporation is carried out to reduce the volume of the mixture by: about 30% to about 70%; about 40% to about 60%, about 45% to about 55%, or about 50%, of the original volume.
65. A process according to Clause 63 or Clause 64, wherein the evaporating is carried out at a temperature of: about 40° C. to about 80° C.; about 45° C. to about 70° C.; about 45° C. to about 60° C.; about 45° C. to about 55° C.; or about 50° C.; preferably wherein the evaporating is carried under reduced pressure, preferably at reduced pressure.
66. A process according to any of Clauses 63, 64, or 65, wherein the cooling is carried to a temperature of: about −10° C. to about 40° C.; about 0° C. to about 35° C.; about 10° C. to about 30° C.; about 20° C. to about 28° C.; or about 25° C.
67. A process according to Clause 59, for preparing Belumosudil Form B6 as defined in any of Clauses 1 to 14, comprising crystallizing Belumosudil from a solvent mixture selected from:
   methanol and tetrahydrofuran (THF);
   methanol and acetic acid; or
   methanol and dimethylsulfoxide (DMSO).
68. A process according to Clause 67, comprising:
   combining Belumosudil with a solvent selected from: THF, acetic acid or DMSO; preferably at temperature of about 20° C. to about 30° C., to form a solution,
   optionally filtering the solution;
   combining the solution with methanol to precipitate Belumosudil;
   optionally seeding; and
   optionally isolating Belumosudil Form B6.
69. A process according to Clause 68, wherein:
   the solvent is THF, and is preferably used in an amount of: about 5 to about 50 ml, about 10 to about 40 ml or about 15 to about 30 ml, or about 20 ml, per gram of Belumosudil; or
   the solvent is acetic acid, and is preferably used in an amount of: about 1 ml to about 15 ml, about 3 ml to about 10 ml; or about 5 m, per gram of Belumosudil; or
   the solvent is DMSO, and is preferably used in an amount of: about 1 ml to about 15 ml, about 2 ml to about 10 ml; or about 3 ml to about 7 ml, per gram of Belumosudil.
70. A process according to Clause 68 or Clause 69, wherein the solution is filtered prior to combining with methanol.
71. A process according to any of Clauses 68, 69 and 70, wherein methanol is added to the solution of Belumusudil in the solvent.
72. A process according to any of Clauses 68, 69, 70, or 71, wherein the methanol is used in an amount of: about 10 ml to about 45 ml, about 15 ml to about 35 m, about 18 to about 32 ml, per gram of Belumosudil.
73. A process according to any of Clauses 68, 69, 70, 71, or 72, comprising seeding the mixture.
74. A process according to any of Clauses 68, 69, 70, 71, 72, or 73, comprising stirring the mixture, preferably at a temperature of: about −10° C. to about 40° C.; about 0° C. to about 35° C.; about 10° C. to about 30° C.; about 20° C. to about 28° C.; or about 25° C.
75. A process according to Clause 74, wherein the mixture is stirred for: about 30 minutes to about 24 hours; about 30 minutes to about 10 hours, about 45 minutes to about 5 hours, about 60 minutes to about 200 minutes.
76. A process according to any of Clauses 68, 69, 70, 71, 72, 73, 74, or 75, comprising isolating the Belumosudil, preferably by filtration.
77. A process according to any of Clauses 68, 69, 70, 71, 72, 73, 74, or 75, comprising isolating and drying the Belumosudil, preferably wherein the drying is carried out at a temperature of: about 40° C. to about 80° C.; about 45° C. to about 70° C.; about 45° C. to about 65° C., preferably at reduced pressure.
78. A process according to Clause 77, wherein the drying is carried out over a period of: about 1 hour to about 36 hours, about 4 hours to about 25 hours, about 6 hours to about 18 hours, about 8 hours to about 15 hours.
79. A process for the preparation of Belumosudil Mesylate, preferably Form M1, wherein the process comprises crystallising Belumosudil Mesylate from a solvent comprising: methanol, or aqueous methanol.
80. A process according to Clause 79, comprising:
   (I) preparing a mixture comprising Belumosudil mesylate in solvent selected from methanol, or methanol and water, and optionally seeds of Belumosudil mesylate, preferably seeds of Belumosudil mesylate form M1;
   (II) optionally cooling the mixture;
   (III) optionally stirring the mixture;
   (IV) optionally isolating the Belumosudil Mesylate; and
   (V) optionally drying.
81. A process according to Clause 79 or Clause 80, wherein the mixture is at a temperature of about −15° C. to about 30° C.; or about −15° C. to about 20° C. or about −15° C. to about 10° C.; or about −15° C. to about 0° C. or about −5° C. to about 30° C.; or about 5° C. to about 30° C.; or about 15° C. to about 30° C.; or about −15° C. to about 20° C.; or about −5° C. to about 20° C.; or about 5° C. to about 20° C.
82. A process according to any of Clauses 79, 80 or 81, wherein the mixture is step (I) is prepared by combining, in any order, Belumosudil and methanesulfonic acid, and optionally Belumosudil mesylate seeds, more preferably wherein the Belumosudil mesylate seeds are Form M1, in the solvent,
83. A process according to any of Clauses 79, 80, 81, or 82, wherein the mixture is cooled in step (II), preferably wherein the cooling is to about 15° C. to about −10° C.
84. A process according to any of Clauses 80, 81, 82, or 83, wherein the mixture is stirred in step (III), preferably for a period of: about 30 minutes to about 10 hours, about 1 hour to about 8 hours, about 1 hour to about 6 hours, or about 1 to about 4 hours.
85. A process according to any of Clauses 80, 81, 82, 83, or 84, wherein the Belumosudil mesylate is isolated in step (IV), preferably by filtration, centrifuge, or decantation, and more preferably by filtration.
86. A process according to any of Clauses 80, 81, 82, 83, 84, or 85, for the preparation of Belumosudil Mesylate Form M1.
87 A process according to any of Clauses 24 to 86, further comprising combining the Belumosudil Mesylate, preferably Belumosudil Mesylate Form M1; or Belumosudil, preferably Belumosudil Form B6, with at least one pharmaceutically acceptable excipient to form a pharmaceutical composition.

1. A crystalline form of Belumosudil, which may be designated form B6, which is characterized by an X-ray powder diffraction pattern having peaks at 7.9, 13.0, 13.6, 20.1 and 26.3 degrees 2-theta±0.2 degrees 2-theta.

2. The crystalline form of Belumosudil according to claim 1, which is characterized by an X-ray powder diffraction pattern having peaks at 7.9, 13.0, 13.6, 20.1 and 26.3 degrees 2-theta±0.2 degrees 2-theta, and also having any one, two, three or four additional peaks selected from 15.3, 16.0, 23.1 and 31.2 degrees 2-theta±0.2 degrees 2-theta.

3. The crystalline form of Belumosudil according to claim 1, which is characterized by an X-ray powder diffraction pattern having peaks at 7.9, 13.0, 13.6, 15.3, 16.0, 20.1, 23.1, 26.3 and 31.2 degrees 2-theta±0.2 degrees 2-theta; or an X-ray powder diffraction pattern substantially as depicted in FIG. 1 or FIG. 2.

4. The crystalline form of Belumosudil according to claim 1, which is a methanol solvate, containing about 6% to about 8% by weight of methanol.

5. (canceled)

6. The crystalline form of Belumosudil according to claim 1, which is substantially free of any other solid state forms; containing about 20% (w/w) or less of any other forms of Belumosudil.

7. A pharmaceutical composition comprising a crystalline form of Belumosudil according to claim 1.

8. (canceled)

9. The pharmaceutical composition of claim 7, further comprising at least one pharmaceutically acceptable excipient.

10. A process for preparing a pharmaceutical formulation, comprising combining the crystalline form of Belumosudil according to claim 1, with at least one pharmaceutically acceptable excipient.

11. The crystalline form of Belumosudil according claim 1, for use as a medicament.

12. (canceled)

13. A method of treating Chronic Graft-Versus-Host Disease, Systemic Sclerosis, Idiopathic pulmonary fibrosis and/or Psoriasis vulgaris, comprising administering a therapeutically effective amount of a crystalline form of Belumosudil according to claim 1 to a subject in need of the treatment.

14. (canceled)

15. A process for the preparation of another solid state or crystalline polymorph form of: Belumosudil or Belumosudil salts or solid state forms thereof, comprising: preparing at least one of an acid addition salt of Belumosudil, Belumosudil mesylate, or crystalline forms of Belumosudil mesylate using the crystalline form of Belumosudil of claim 1.

16. A process for preparing other solid state forms of Belumosudil, Belumosudil salts and their solid state forms thereof comprising:
preparing the crystalline form of Belumosudil according to claim 1; and
converting the crystalline form of Belumosudil to a different form of Belumosudil or to a salt Belumosudil including at least one of an acid addition salt of Belumosudil or Belumosudil mesylate.

17. The process according to claim 16, comprising reacting the crystalline form of Belumosudil with methanesulfonic acid, and optionally isolating Belumosudil Mesylate.

18. A process for the preparation of Belumosudil, the process comprising:
(a) reacting 1,1-dimethylethyl 5-[[2-[3-[2-[(1-methylethyl)amino]-2-oxoethoxy]phenyl]-4-quinazolinyl]amino]-1H-indazole-1-carboxylate with a base; and
(b) optionally isolating Belumosudil.

19. The process according to claim 18, wherein the Belumosudil is characterized by an X-ray powder diffraction pattern having peaks at 7.9, 13.0, 13.6, 20.1 and 26.3 degrees 2-theta±0.2 degrees 2-theta.

20. (canceled)

21. The process according to claim 18, wherein step (a) is carried out in one or more polar solvents, or wherein step (a) is carried out in one or more polar protic solvents including one or more alcohols, wherein the alcohol is a C1 to C6 alcohol, or a C1-C3 aliphatic alcohol, or methanol; or wherein step (a) is carried out in a mixture of one or more alcohols, wherein the alcohol is at least one of a C1 to C6 alcohol, or a C1-C3 aliphatic alcohol, or methanol and a polar aprotic solvent including at least one of DMSO, THF, or DMF.

22. The process according to claim 18 wherein step (a) is carried out in methanol; or a mixture of methanol and DMSO.

23. The process according to claim 18, wherein wherein step (a) is carried out in one or more solvents, which is a mixture of methanol and DMSO, and the reaction is carried out at a temperature of 20° C. to about 50° C.

24. The process according to claim 29, wherein the amount of cesium carbonate in step (a) is about 0.12 mole equivalents to about 1 equivalent, 0.4 equivalents to about 0.6 equivalents relative to 1,1-dimethylethyl 5-[[2-[3-[2-[(1-methylethyl)amino]-2-oxoethoxy]phenyl]-4-quinazolinyl]amino]-1H-indazole-1-carboxylate.

25. The process according to claim 18, wherein step (b) comprises precipitating the Belumosudil, optionally comprising addition of seed crystals of Belumosudil.

26. The process according to claim 18, wherein step (b) comprises combining a reaction mixture of step (b) with methanol and wherein step (b) is carried out at a temperature of about 10° C. to about 35° C.

27. The process according to claim 18, wherein the base in step (a) is an inorganic base.

28. The process according to claim 18, wherein the base in step (a) is a carbonate salt.

29. The process according to claim 18, wherein the base in step (a) is cesium carbonate.
